(19) United States
(12) Patent Application Publication
(10) Pub. No.: US 2022/0096687 A1
(43) Pub. Date: Mar. 31, 2022

(54) Sanitizer Aerosol Dispensing Apparatus

(71) Applicant: Tabitha Angel Bryant, Columbia, SC (US)

(72) Inventor: Tabitha Angel Bryant, Columbia, SC (US)

(21) Appl. No.: 17/217,966

(22) Filed: Mar. 30, 2021

Related U.S. Application Data
(63) Continuation-in-part of application No. 29/753,006, filed on Sep. 29, 2020.

(51) Int. Cl. A61L 2/22; A61L 9/14

(52) U.S. Cl. CPC A61L 2/22; A61L 2209/133; A61L 2202/11; A61L 9/14

(57) ABSTRACT

A sanitizer aerosol dispensing apparatus suited for disinfecting enclosed areas is presented. The sanitizer aerosol dispensing apparatus contains a dispenser canister, a dispensing mechanism, and a weighted base. The dispenser canister contains a top portion, a bottom portion, and a liquid reservoir. The liquid reservoir is positioned within the dispenser canister. The dispensing mechanism is connected adjacent to the dispenser canister. The dispensing mechanism is in fluid communication with the liquid reservoir, where the dispensing mechanism is configured to dispense a fluid from the liquid reservoir. The weighted base is positioned adjacent to the bottom portion, opposite to the top portion.

### FIELD OF THE INVENTION

The present invention relates to sanitizing devices. More specifically, the present invention is a sanitizer aerosol dispensing device suited for disinfecting enclosed areas.

### BACKGROUND OF THE INVENTION

Sanitation measures are often employed at sites as a means to provide health safety in facilities. Sanitation specialists may be deployed to a site disinfect an area. These sanitation specialists oftentimes employ sprayers and misters that are bulky and unwieldy. Therefore, it is an objective of the present invention to provide a handheld safety device that can effectively sterilize and disinfect an area of interest.

The present invention aims to provide a better solution. The present invention is a handheld sanitizing device that effectively disinfects an enclosed area. Additionally, the present invention may be deployed remotely or thrown mitigating exposure to the user.

### SUMMARY OF THE INVENTION

The present invention is a sanitizer aerosol dispensing apparatus suited for disinfecting enclosed areas. The sanitizer aerosol dispensing apparatus comprises a dispenser canister, a dispensing mechanism, and a weighted base. The dispenser canister comprises a top portion, a bottom portion, and a liquid reservoir. The liquid reservoir is positioned within the dispenser canister. The dispensing mechanism is connected adjacent to the dispenser canister. The dispensing mechanism is in fluid communication with the liquid reservoir, where the dispensing mechanism is configured to dispense a fluid from the liquid reservoir. The weighted base is positioned adjacent to the bottom portion, opposite to the top portion.

### BRIEF DESCRIPTION OF THE DRAWINGS

FIG. 1 is a perspective view of the present invention.

FIG. 2 is a top perspective exploded view of the present invention.

FIG. 3 is a bottom perspective exploded view of the present invention.

FIG. 4 is a fluid diagram used in the present invention.

### DETAIL DESCRIPTIONS OF THE INVENTION

All illustrations of the drawings are for the purpose of describing selected versions of the present invention and are not intended to limit the scope of the present invention. The present invention is to be described in detail and is provided in a manner that establishes a thorough understanding of the present invention. There may be aspects of the present invention that may be practiced or utilized without the implementation of some features as they are described. It should be understood that some details have not been described in detail in order to not unnecessarily obscure focus of the invention. References herein to “the preferred embodiment”, “one embodiment”, “some embodiments”, or “alternative embodiments” should be considered to be illustrating aspects of the present invention that may potentially vary in some instances, and should not be considered to be limiting to the scope of the present invention as a whole.

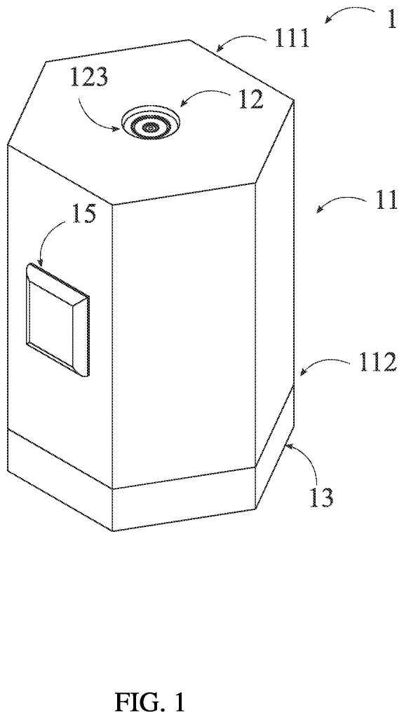

In reference to FIGS. 1-3, the present invention is a sanitizer aerosol dispensing apparatus 1. In the preferred embodiment of the present invention, the sanitizer aerosol dispensing apparatus 1 resembles a handheld fogger device that allows a user to disinfect an enclosed area safely and easily. The sanitizer aerosol dispensing apparatus 1 comprises a dispenser canister 11, a dispensing mechanism 12, and a weighted base 13. The dispenser canister 11 comprises a top portion 111, a bottom portion 112, and a liquid reservoir 113. The liquid reservoir 113 is positioned within the dispenser canister 11. The dispensing mechanism 12 is connected adjacent to the dispenser canister 11. The dispensing mechanism 12 is in fluid communication with the liquid reservoir 113, where the dispensing mechanism 12 is configured to dispense a fluid from the liquid reservoir 113. The weighted base 13 is positioned adjacent to the bottom portion 112, opposite to the top portion 111. In the preferred embodiment of the present invention, the sanitizer aerosol dispensing apparatus 1 is made out of any suitable material, such as, but not limited to nylon composite, aluminum, or any other suitable material. In the preferred embodiment of the present invention, the sanitizer aerosol dispensing apparatus 1 is hexagonal in shape. In another embodiment of the present invention, the sanitizer aerosol dispensing apparatus 1 is rectangular in shape. In some embodiments of the present invention, the sanitizer aerosol dispensing apparatus 1 is spherical in shape. In various embodiments, the sanitizer aerosol dispensing apparatus 1 may take the form of any shape. In another embodiment of the present invention, the sanitizer aerosol dispensing apparatus 1 is scaled to any size, such that the sanitizer aerosol dispensing apparatus 1 serves as a large-scale fogger suitable for large areas. In the preferred embodiment of the present invention, the dispensing mechanism 12 may take the form of any suitable implement of dispensing a sanitizing solution into an aerosol form, such as, but not limited to electropneumatic misters, aerosol misters, atomizers, or any other suitable dispensing implement. In the preferred embodiment of the present invention, the weighted base 13 may take the form of a counterweight that assists in propping up the sanitizer aerosol dispensing apparatus 1 in a vertical position, such that the nozzle 123 is projected upwards.

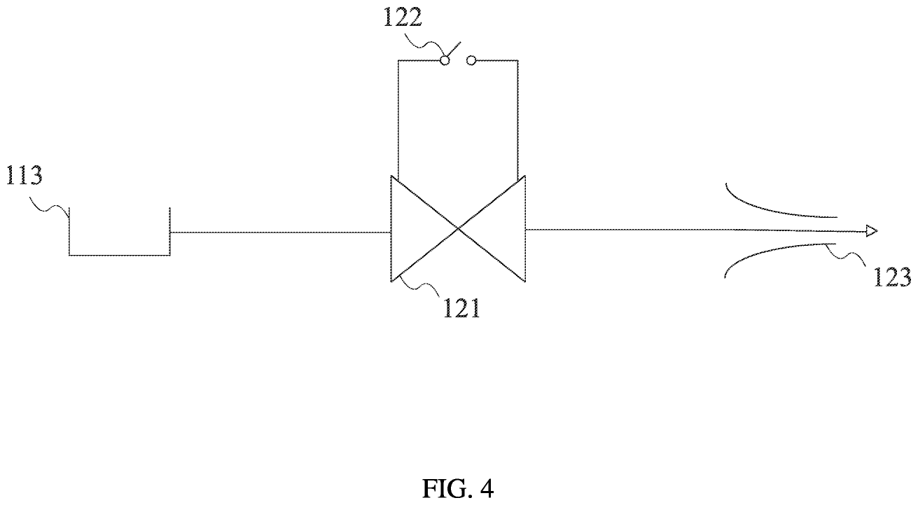

In reference to FIGS. 1-4, the dispensing mechanism 12 comprises a release valve 121, a switch 122, and a nozzle 123. The release valve 121 is in fluid communication between the nozzle 123 and the liquid reservoir 113. The switch 122 is operatively engaged to the release valve 121, where the switch 122 is configured to actuate the release valve 121 to dispense a fluid from the liquid reservoir 113. In the preferred embodiment of the present invention, the release valve 121 may take the form of any suitable valve implement, such as, but not limited to solenoids, mechanical valves, or any other suitable valve implement. In the preferred embodiment of the present invention, the switch 122 may take the form of an actuating implement that activates the release valve 121. The switch 122 may take the form of a push button type actuator but may take the form of any other suitable actuator, such as, but not limited to remote actuators, toggle actuators, impact activated actuators, or any other suitable actuator implement. In one embodiment of the present invention, the switch 122 is a timer activated switch 122, where the switch 122 is configured to activate the release valve 121 at a set time. In one embodiment of the present invention, the switch is a toggle switch 122 where the switch 122 is configured to activate the release valve 121 at a set emergency condition. More specifically, this emergency condition may take the form of an emergency lockdown protocol, panic button, or any other suitable type of emergency condition. In the preferred embodiment of the present invention, the nozzle 123 is recessed within the dispenser canister 11. This secures the nozzle 123 within the dispenser canister 11, preventing external damage to the nozzle 123 when the sanitizer aerosol dispensing apparatus 1 is tossed or handled. In the preferred embodiment of the present invention, the nozzle 123 is connected adjacent to the top portion 111. This orients the nozzle 123 upwards along the top portion 111 as the base props up the sanitizer aerosol dispensing apparatus 1.

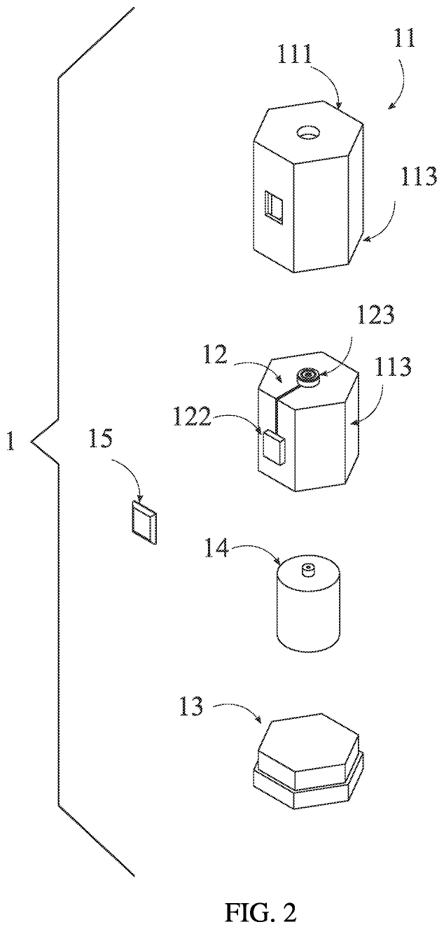

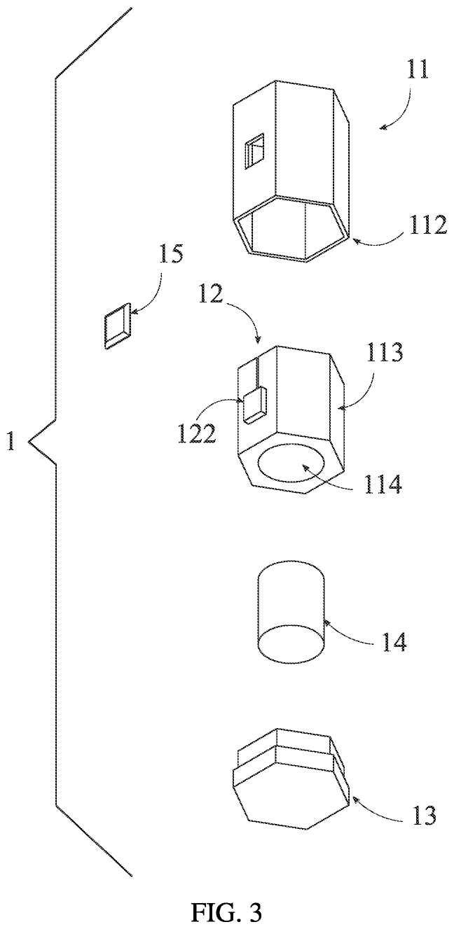

In another embodiment, the sanitizer aerosol dispensing apparatus 1 further comprises a reservoir insert 14. The liquid reservoir 113 comprises an insert receiver 114, as shown in FIGS. 2-3. The insert receiver 114 is positioned within the liquid reservoir 113. The reservoir insert 14 is removably attached to the insert cavity. The reservoir insert 14 is in fluid communication with the dispensing mechanism 12. The reservoir insert 14 is in fluid communication with the release valve 121. In this embodiment, the sanitizer aerosol dispensing apparatus 1 is refillable through the user of the replaceable reservoir insert 14. The sanitizer aerosol dispensing apparatus 1 further comprises a cap 15, as shown in FIGS. 1-3. The cap 15 is removably attached to the switch 122, where the cap 15 is configured to secure the switch 122 from unintended activation.

Although the invention has been explained in relation to its preferred embodiment, it is to be understood that many other possible modifications and variations can be made without departing from the spirit and scope of the invention as hereinafter claimed.

What is claimed is:

1. A sanitizer aerosol dispensing apparatus comprising:
a dispenser canister;
a dispensing mechanism;
a weighted base;
the dispenser canister comprising a top portion, a bottom portion, and a liquid reservoir;
the liquid reservoir being positioned within the dispenser canister;
the dispensing mechanism being connected adjacent to the dispenser canister;
the dispensing mechanism being in fluid communication with the liquid reservoir, wherein the dispensing mechanism is configured to dispense a fluid from the liquid reservoir; and
the weighted base being positioned adjacent to the bottom portion, opposite to the top portion.

2. The sanitizer aerosol dispensing apparatus as claimed in claim 1 comprising:
the dispensing mechanism comprising a release valve, a switch, and a nozzle;
the release valve being in fluid communication between the nozzle and the liquid reservoir; and
the switch being operatively engaged to the release valve, wherein the switch is configured to actuate the release valve to dispense a fluid from the liquid reservoir.

3. The sanitizer aerosol dispensing apparatus as claimed in claim 1 comprising:
the dispensing mechanism comprising a nozzle; and
the nozzle being recessed within the dispenser canister.

4. The sanitizer aerosol dispensing apparatus as claimed in claim 3 comprising:
the nozzle being connected adjacent to the top portion.

5. The sanitizer aerosol dispensing apparatus as claimed in claim 1 comprising:
a reservoir insert;
the dispenser canister comprising an insert receiver;
the insert receiver being positioned within the liquid reservoir;
the reservoir insert being removably attached to the insert cavity; and
the reservoir insert being in fluid communication with the dispensing mechanism.

6. The sanitizer aerosol dispensing apparatus as claimed in claim 2 comprising:
a reservoir insert;
the liquid reservoir comprising an insert receiver;
the insert receiver being positioned within the liquid reservoir;
the reservoir insert being removably attached to the insert cavity; and
the reservoir insert being in fluid communication with the release valve.

7. The sanitizer aerosol dispensing apparatus as claimed in claim 2 comprising:
a cap; and
the cap being removably attached to the switch, wherein the cap is configured to secure the switch from un-intended activation.

8. The sanitizer aerosol dispensing apparatus as claimed in claim 1, wherein the sanitizer aerosol dispensing apparatus is hexagonal in shape.

9. The sanitizer aerosol dispensing apparatus as claimed in claim 1, wherein the sanitizer aerosol dispensing apparatus is rectangular in shape.

10. The sanitizer aerosol dispensing apparatus as claimed in claim 1, wherein the sanitizer aerosol dispensing apparatus is spherical in shape.

11. The sanitizer aerosol dispensing apparatus as claimed in claim 2, wherein the switch is a timer activated switch.

12. The sanitizer aerosol dispensing apparatus as claimed in claim 2, wherein the switch is a toggle switch.
